(19) United States
(12) Patent Application Publication
(10) Pub. No.: US 2022/0290090 A1
(43) Pub. Date: Sep. 15, 2022

(54) AUTOMATED CONTROL AND PREDICTION FOR A FERMENTATION SYSTEM

(71) Applicant: Culture Biosciences, Inc., South San Francisco, CA (US)

(72) Inventors: Matthew Adams BALL, San Mateo, CA (US); William Graham PATRICK, San Francisco, CA (US); Collin David James EDINGTON, San Mateo, CA (US)

(21) Appl. No.: 17/721,556

(22) Filed: Apr. 15, 2022

Related U.S. Application Data
(63) Continuation of application No. PCT/US2020/058932, filed on Nov. 4, 2020.
(60) Provisional application No. 62/930,848, filed on Nov. 5, 2019.

(51) Int. Cl. C12M 1/21; C12M 1/36; C12M 1/34; G05D 21/02; C12M 1/12

(52) U.S. Cl. CPC C12M 41/02; C12M 41/48; C12M 41/26; C12M 41/36; C12M 41/42; G05D 21/02; C12M 37/02

(57) ABSTRACT

The present disclosure provides methods and systems for foam control. A method of foam control for a fermentation system comprises: obtaining image data from an imaging device located at the fermentation system; and processing said sensor data using a trained machine learning algorithm to generate an output that indicates presence of foam or level of foaming.

### CROSS REFERENCE

This application is a continuation of International Application No. PCT/US2020/058932, filed Nov. 4, 2020, which claims priority to U.S. Provisional Application No. 62/930,848, filed Nov. 5, 2019, each of which are incorporated herein by reference in their entirety.

### BACKGROUND OF THE INVENTION

Large-scale fermentation processes can be used to produce healthcare products, food additives, alcohol, enzymes, biofuels, agricultural treatments, and industrial chemicals. During the fermentation process, microorganisms can be used to produce antibiotics, diagnostics, therapeutics, food products, chemicals, and biofuels. In the case of microbiome therapeutics or microbial agricultural treatment, the organisms themselves can be the product. Due to the importance of fermentation processes, an ability to monitor and control fermentation processes in an automated fashion could be valuable for the healthcare, food science, and biotechnology industries.

### INCORPORATION BY REFERENCE

Each patent, publication, and non-patent literature cited in the application is hereby incorporated by reference in its entirety as if each was incorporated by reference individually.

### SUMMARY OF THE INVENTION

The present disclosure provides systems and methods for generating estimations and/or predictions of important parameters during a fermentation process. In some embodiments, the method may implement machine learning algorithm to generate predictive models and forecasts that can be used to predict, for example, the status and result of fermentation processes run on a system disclosed herein. In particular, the provided methods and systems may be capable of detecting and predicting presence of foaming and/or foaming level using a machine learning-based detection mechanism. In some cases, foaming and/or other parameters of the fermentation process may be automatically controlled based on an estimated state of the process.

Additional aspects and advantages of the present disclosure will become readily apparent to those skilled in this art from the following detailed description, wherein only exemplary embodiments of the present disclosure are shown and described, simply by way of illustration of the best mode contemplated for carrying out the present disclosure. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example of a bioreactor, in accordance with embodiments of the invention.

FIG. 2 shows an example of a fermentation automation workcell, in accordance with embodiments of the invention.

FIG. 3 shows an example of a bay of a bioreactor array, in accordance with embodiments of the invention.

FIG. 4 shows an exemplary process of processing image data using a trained model for foam detection and prediction.

FIG. 5 shows examples of images captured by an imaging device for foaming control.

FIG. 6 schematically illustrates a fermentation system implementing foam detection and prediction mechanism.

FIG. 7 schematically shows block diagrams of processing sensor data using a trained model for state estimation.

FIG. 8 schematically illustrates a monitoring system for monitoring and controlling a fermentation process.

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

The present disclosure provides systems and methods for monitoring and estimating a state of a fermentation process. In some embodiments, one or more operational parameters of the fermentation process may be adjusted based on the estimated state thereby controlling the fermentation process in an automated fashion. In one aspect, the state of the fermentation process may include presence of foam and/or the detection of an amount/level of foam. The state can also include events that may indicate a stage in a normal fermentation process or undesired results such as contamination. The state may be experiment-specific or organism-specific.

Foaming is a serious problem for biochemical processes. Foam can be produced as an unwanted consequence in the manufacture of various substances such as surfactants and proteins, particularly in processes involving significant shear forces near air-liquid interfaces, such as those involving aeration, pumping or agitation. Aerobic submerged fermentation relies on adequate aeration to supply oxygen required by the microorganisms to grow and produce product of interest. The introduction of air into the fermentation broth to provide oxygen required by the microorganism may generate foam. The presence of foam during fermentation generally has negative impacts on its performance, including reduction of fermentor working volume or productivity, and a risk of contamination associated with a defoaming, such as the production of a foam column or foam head above the liquid fermentation broth of sufficient height that it exits the fermentation vessel through venting or pipes.

In some cases, in response to detection of presence of foam or the level/amount of foam, one or more actions may be performed automatically to reduce or mitigate foaming. For instance, additives such as antifoam or defoamers may be added in the appropriate doze or amount to mitigate foam formation based on the detected amount/degree of foam. In some cases, the presence of foam may be predicted such that one or more actions may be performed to prevent foaming from happening. The one or more actions may include operations to change the process conditions such that antifoam agent may not be needed. An early intervention to prevent foaming may advantageously reduce the amount of antifoam agents added to the bioreactor. This may beneficially reduce contaminations introduced by additive of the antifoam agents.

As used herein, the term “process conditions” generally refers to a solvent and/or a choice of physical parameters such as, but not limited to, temperature, pressure, mixing or pH involved in the methods of the present invention.

As used herein, the term “foam” generally refers to a substance that is formed by trapping gaseous bubbles in a liquid, in a gel or in a semisolid.

In one aspect, the present disclosure provides systems and methods for detecting and predicting foaming based on image data. In particular, the provided systems and methods may utilize machine learning algorithm trained models to detect and predict the presence or level/degree of foaming. Alternatively or in addition to, a trained model may be used for foam control. The output of the trained model may be control signals/commands to a controller of the automated fermentation system such that one or more parameters or process conditions may be adjusted.

In an aspect, a method for foam control for a fermentation system is provided. The method comprises: obtaining image data from an imaging device located at the fermentation system; and processing the image data using a first machine learning algorithm trained model to generate an output indicating a presence of foam or a level of foaming within a bioreactor of the fermentation system. In some embodiments, the fermentation system comprises a plurality of the bioreactors configured to receive a fermentation agent and a controller for controlling one or more components of each of the plurality of the bioreactors. In some cases, the one or more components are controlled based on a control instruction generated based at least in part on the presence of foam or the level of foaming. In some embodiments, the image data contains at least a portion of a content in the bioreactor.

In some embodiments, the method further comprises generating a control signal to adjust an operational status of the bioreactor based on the presence of foam or the level of foaming. In some cases, the operational status is selected from airflow, pressure, temperature, or an agitation speed within the bioreactor.

In some embodiments, the method further comprises predicting, using a second machine learning algorithm trained model, a state of a fermentation process within the bioreactor based at least in part on the image data and one or more real-time parameters. In some cases, the one or more real-time parameters are estimated using sensor data. For example, at least a portion of the sensor data is not a direct measurement of the one or more real-time parameters. In some cases, the one or more real-time parameters are pH, dissolved oxygen tension, optical density, or temperature. In some embodiments, the fermentation system comprises a robotic component configured to provide a fermentation agent to the bioreactor.

In a separate yet related aspect, a system for foam control in a fermentation system is provided. The system comprises: an imaging device located at the fermentation system, wherein the imaging device is configured to capture image data containing at least a portion of a bioreactor of the fermentation system; and one or more processors configured to: receive the image data and process the image data using a first machine learning algorithm trained model to generate an output indicating a presence of foam or a level of foaming within the bioreactor.

In some embodiments, the fermentation system comprises a plurality of the bioreactors configured to receive a fermentation agent and a controller for controlling one or more components of each of the plurality of the bioreactors. In some cases, the one or more components are controlled based on a control instruction generated based at least in part on the presence of foam or the level of foaming.

In some embodiments, the image data contains at least a portion of a content in the bioreactor. In some embodiments, the one or more processors are configured to further generate a control command to adjust an operational status of the bioreactor based on the presence of foam or the level of foaming. In some cases, the operational status is selected from the group consisting of airflow, pressure, temperature and agitation speed within the bioreactor.

In some embodiments, the one or more processors are configured to further predict, using a second machine learning algorithm trained model, a state of a fermentation process within the bioreactor based at least in part on the image data and one or more real-time parameters. In some cases, the one or more real-time parameters are estimated using sensor data. For example, at least a portion of the sensor data is not a direct measurement of the one or more real-time parameters. In some cases, the one or more real-time parameters are selected from the group consisting of pH, dissolved oxygen tension, optical density and temperature. In some embodiments, the fermentation system comprises a robotic component configured to provide a fermentation agent to the bioreactor.

The present disclosure provides systems and methods for fermentation with improved control capability. Various aspects of the invention described herein can be applied to any of the particular applications set forth below. The invention can be applied as a standalone system for monitoring and controlling foaming, or monitoring and controlling one or more parameters of a fermentation process. Alternatively or in addition to, the invention can be an integral part of a fermentation automation work cell, or an integrated system for data collection and analysis. Different aspects of the invention can be appreciated individually, collectively, or in combination with each other.

### Automated Fermentation System

An automated fermentation system disclosed herein can be of any size. For example, the automated fermentation system can be the size of a facility, a room, a car, a benchtop, or can be a handheld or portable system. The enclosure can enclose the space of a facility, a room, a car, a benchtop, or can be a handheld or easily transportable item. In some instances, the system can be larger than, approximately the same size as, or smaller than a shipping container. One or more dimensions of the system (for example, length, width, height, diagonal, diameter) can be less than or equal to about 1 cm, about 2 cm, about 3 cm, about 5 cm, about 10 cm, about 20 cm, about 50 cm, about 1 m, about 1.5 m, about 2 m, about 3 m, about 4 m, about 5 m, about 7 m, about 10 m, about 12 m, about 15 m, about 20 m, about 25 m, about 30 m, about 35 m, about 40 m, about 50 m, about 75 m, or about 100 m. One or more dimensions of the system can be greater than any of the values provided, or fall within a range between any two of the values provided. The enclosure can have one or more dimensions less than any of the values provided. One or more dimensions of the enclosure can be greater than any of the values provided or fall within a range between any two of the values provided. In some embodiments, a maximum dimension of the system or enclosure (greatest of length, width, or height) can have a value less than any of the values provided, greater than any of the values provided, or falling within a range between any two of the values provided.

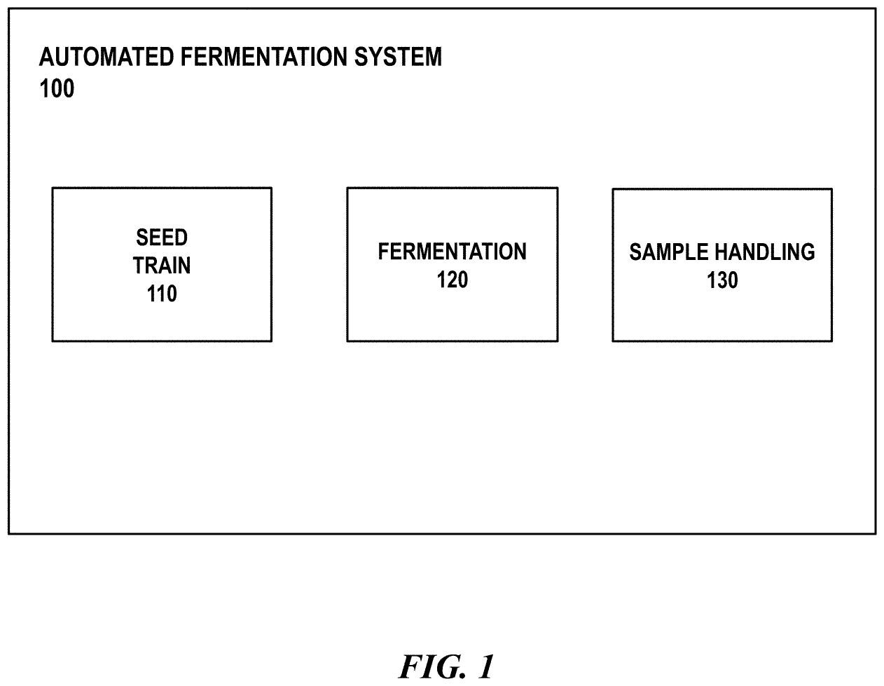

One or more processes within the automated fermentation system can be fully automated. One or more processes within the enclosure can be fully automated. A process can be automated and executed without requiring human intervention. A process can be automated when a human does not need to perform any manual manipulation. A process can be automated with the aid of one or more processors and one or more sensors. A process can be automated if the presence of a human is not required within an enclosure of the automated fermentation system. In some embodiments, seed train preparation 110, fermentation 120, and/or sample handling 130 can be fully automated as shown in FIG. 1. In some embodiments, transfer of materials from a seed train station to a fermentation station can be fully automated. A seed train can refer to a process by which a sufficient number of fermentation agents are produced to inoculate the bioreactors. A seed train process can start with the thawing of a cryopreserved cell bank vial, followed by multiple culturing steps in progressively larger culture vessels.

In some embodiments, transfer of materials from a fermentation station to a sample handling station can be fully automated. In some embodiments, one or more preparation processes prior to fermentation can be automated. A fermentation process can be automated. One or more parameters or states of the fermentation process can be monitored and controlled by employing a machine-learning-based mechanism. Sample handling after fermentation can be automated. Sample handling can include sample preparation and/or analysis. The provided machine-learning-based monitoring and control mechanism can be applied to the seed train preparation 110, fermentation 120, or sample handling 130 stage.

In some embodiments, one or more robotic components can aid in an automated process described herein. One or more robotic components can comprise one or more robotic arms. A description herein of a robotic arm can apply to any type of robot or robotic component. For example, any description of an arm can apply to a gantry, such as a three-axis gantry. A robotic arm can be capable of interacting with a seed train preparation station, a fermentation station, and/or a sample handling station. A robotic arm can aid in transfer of materials within a seed train preparation station, within a fermentation station, and/or within a sample handling station. A robotic arm can be capable of aiding in the transfer of materials between a seed train preparation station and a fermentation station, or between a fermentation station and a sample handling station.

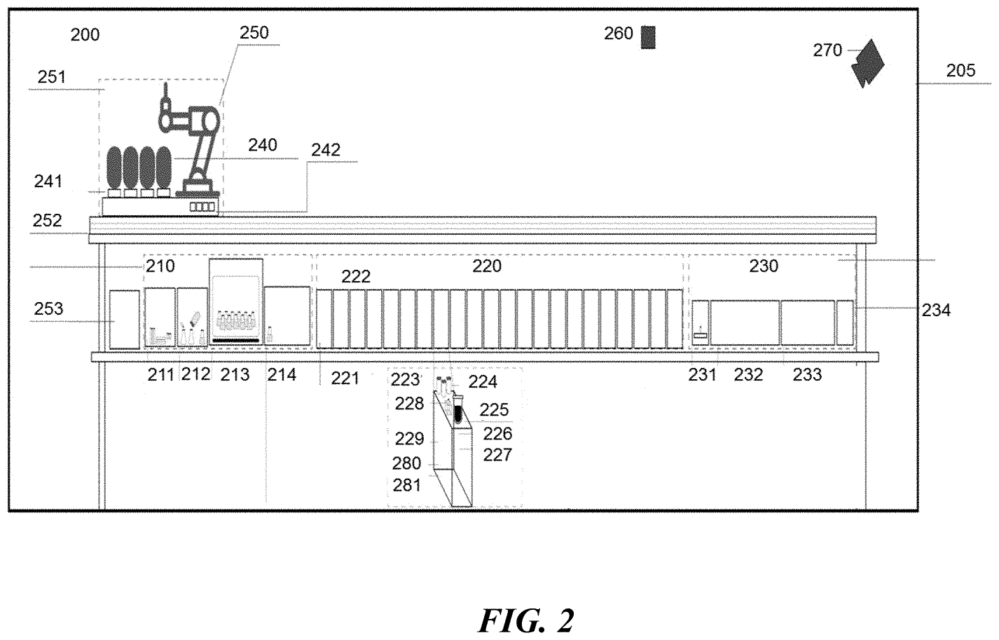

FIG. 2 shows an example of a fermentation automation workcell 200, in accordance with embodiments of the invention. A workcell may be an automated fermentation system. A workcell may comprise a sterile enclosure 205. A workcell may comprise an automated seed train station 210, a fermentation station 220, and/or a sample handling station 230. Sample preparation and analysis is performed at the sample handling station 230. A workcell may also comprise one or more robotic components. Any description herein of a robot may apply to a robotic arm or other type of robotic component.

A workcell may comprise an automated seed train station 210, a fermentation station 220, and/or a sample handling station 230. Sample preparation and analysis is performed at the sample handling station 230. A workcell may also comprise one or more robotic components. Any description herein of a robot may apply to a robotic arm or other type of robotic component.

Any station described herein may or may not comprise a physical region within the workcell. A station may be spread out over multiple locations within a workcell. A station may be localized to a single location or region within a workcell. One or more components of a station may interact with one another. One or more components of a station may operate independently of one another. In some embodiments, one or more components of a station may operate in series, or in parallel.

A seed train station 210 may permit strain input 211, seed preparation 212, incubation 213, and/or inoculation 214. Optionally, a storage station, such as a cold storage station, may be provided, which may permit strains to be stored before they are used. Such activities may occur in the order provided or in any other order. Any of the processes may be optional or additional processes may be included. Any activities at a seed train station may be automated. In some embodiments, all activities at a seed train station may be automated. For instance, activities, such as strain input, seed preparation, shaker incubation, and/or inoculation may be performed automatically without requiring human intervention. One or more of the activities may be performed with aid of a robot.

Any activity at a seed train station may be monitored. For instance, seeds may be sampled as they are growing, and data about seeds may be collected. Data about seeds in the seed train station may be collected with aid of one or more sensors. The one or more sensors may or may not require the collection of one or more samples.

Media may be provided to the workcell. Media may be provided via one or more containers, such as bulk media bottles 240. The containers may be filled by a human operator and may be brought into the workcell. The filling may occur outside or within the workcell. The bottles may be brought into the bay. A robot may dispense the media into media bottles on the bay. A robot may dispense the media to a seed train station. Automated media preparation may occur.

Optionally, one or more sensors may be provided. In some embodiments, a sensor may be used to track initial media volume. One or more sensors may be employed to track strain volume or type. One or more sensors may be employed to determine any material quantity (e.g., volume, weight, height, density, concentration, or other measurement).

During incubation 213, containers may be added to an incubator. Optionally, incubation may or may not include shaking. Any description herein of shaker incubation may apply to any type of incubation which may or may not include a shaker. A robot may aid in one or more activities during shaker incubation. For instance, a robot may open a shaker/incubator. A robot may add containers, such as flasks or tubes to the shaker/incubator. The containers may contain strain and/or media. Robot may optionally close a shaker/incubator after the containers have been added. A robot may open or close a shaker/incubator with aid of a gripper, magnets, suction, or any other technique. Any number of shakers/incubators may be provided. For instance, a single shaker/incubator, two shakers/incubators, three shakers/incubators, four shakers/incubators, five shakers/incubators, or move may be provided. Each shaker/incubator may be capable of operating independently of one another. Each shaker/incubator may have independent settings that can be adjusted.

In some instances, one or more sensors may be provided. A sensor may provide live optical density (OD) monitoring of each culture. A temperature sensor may be provided within a shaker/incubator. An open close sensor may detect when a shaker/incubator is open/closed. This may be useful for determining whether a door is properly closed when it should be, or when a robot needs to open or close a door. In some embodiments, data from one or more sensors may be used to affect operation of a shaker/incubator. For example, data from an OD sensor and/or temperature sensors may be used as feedback for operation of a shaker/incubator. Alternatively, data from the sensors may not affect operation of the shaker/incubator. For example, optical density may be measured as a data point. Data from one or more sensors may affect operation of a robot. For instance, a robot may be instructed to interact with the shaker/incubator or containers within the shaker/incubator based on data from one or more sensors.

In some cases, a machine learning-based control mechanism may be employed to control the operation of the shaker/incubator. For instance, a trained model may process the input data such as the one or more types of sensor data and the output of the model may be control signals/commands to the shaker/incubator.

During inoculation 214, a robot may transfer an inoculation volume from a container to another container. For example, a robot may transfer an inoculation volume from a flask into a tube. A robot may transfer an inoculation volume from a container that was within the shaker/incubator to a container that will be transferred to a bioreactor. The robot may transfer the inoculation volume using any technique. For example, the robot may pipette the inoculation volume from the first container to the second container. Optionally, the robot may pick up and pour selected volume of the first container into the second container. The inoculation volume may comprise the materials have that undergone shaker incubation. The inoculation volume may comprise strain that has undergone the shaker incubation.

The inoculation volume may be transferred to a bioreactor at a fermentation station. For instance, the second container, such as a tube or any other type of container, may be transferred to a bioreactor. The second container may be a single-use vessel. In some embodiments, the container used at the bioreactor may be disposable. Alternatively, the container may be reusable.

One or more optional sensors may be provided for use during inoculation. For instance, a quantity of the inoculation volume may be measured (e.g., volume, weight, height, density, concentration, or other measurement). For instance, a scale may be employed to measure the inoculation volume. Optionally one or more optical devices may be provided. For example, a barcode reader may be employed to recognize one or more barcodes (e.g., 1D code, 2D code, 3D code, QR code, etc.). An optical device, or scanner, may be capable of reading and recognizing any visual marker. This may aid in identification of the inoculation volume and tracking the presence and/or location of the inoculation volume within the workcell.

A fermentation station 220 may comprise a bioreactor array 221. A bioreactor array may comprise one or more bioreactors 222. The fermentation station 220 may comprise a machine learning-based control mechanism for controlling one or more operational parameters of process conditions associated with the fermentation station. In some cases, a trained model may process the input data such as image data and the output of the model may be control signals/commands to one or more components of the fermentation station to add antifoam agents or adjust one or more parameters to prevent foaming. In some cases, a trained model may process the input data such as the one or more types of sensor data and the output of the model may be an estimated state of the fermentation process.

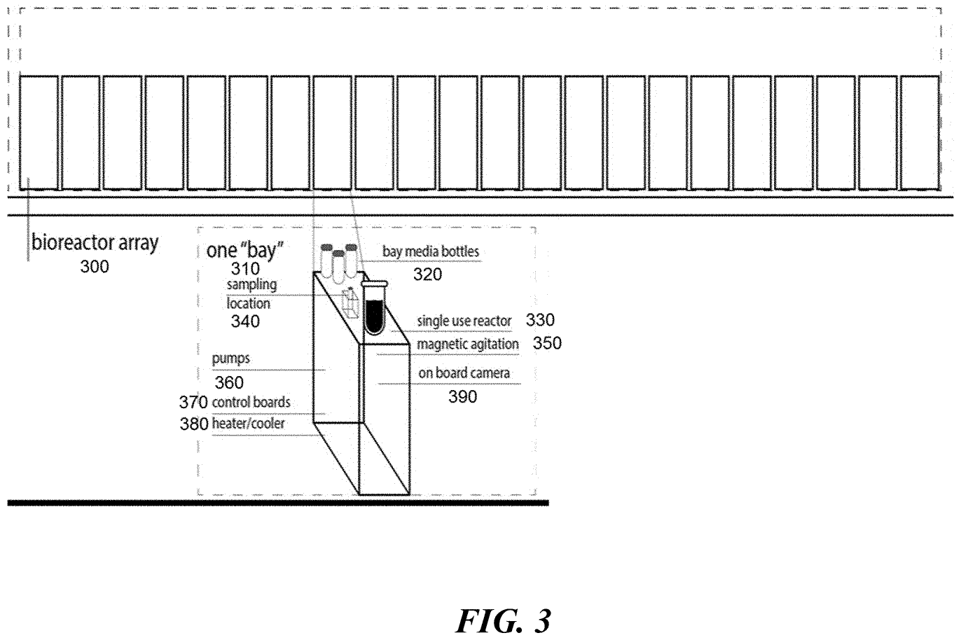

FIG. 3 shows an example of a bay of a bioreactor array 300, in accordance with embodiments of the invention. The bioreactor array may be provided at a fermentation station of a workcell, as discussed elsewhere herein. A bioreactor array may comprise one or more bioreactors 310. The bioreactors may also be referred to as bioreactor bays (or bay), reactor vessels, or bioreactor modules. A fermentation station may comprise a plurality of bioreactors. The bioreactors may be arranged in any fashion. A bioreactor array may comprise a single row of bioreactors, multiple rows of bioreactors, a single column of bioreactors, multiple columns of bioreactors, a single stack of bioreactors, or multiple stacks of bioreactors. A bioreactor array may be an m×n array of bioreactors, or an m×n×p array of bioreactors, where m, n, and p are whole numbers of 1 or greater. Optionally, m, n, or p may be greater than or equal to 1, 2, 3, 4, 5, 6, 7, 8, 9, 10, 12, 15, 20, 30, 40, 50, or 100. Optionally, m, n, or p may be less than any of the number provided or fall within a range between any two of the numbers provided.

Any number of bioreactors may be provided within a bioreactor array. For instance, a bioreactor array may comprise 1 or more, 2 or more, 4 or more, 6 or more, eight or more, 10 or more, 12 or more, 18 or more, 24 or more, 36 or more, 48 or more, 60 or more, 96 or more, 128 or more, 256 or more, or any other number of bioreactors. The bioreactor array may comprise less than any of the numbers provided herein, or fall within a range between any two of the numbers provided herein.

Any number of bioreactor arrays may be provided at a fermentation station. For instance, a single bioreactor array may be provided at a fermentation station. Alternatively, a plurality of bioreactor arrays (e.g., two or more, three or more, four or more, five or more) bioreactor arrays may be provided at a fermentation station.

In some embodiments, each bioreactor within a bioreactor array may be capable of operating independently of other bioreactors within the bioreactor array. Each bioreactor array may be capable of operating independently of other bioreactor arrays.

A single robot may serve a single bioreactor. Alternatively, a single robot may serve multiple bioreactors. In some instances, a single robot may serve at least 1, 2, 4, 6, 8, 12, 18, 24, 36, 48, 96, 128, 256, or more bioreactors. Alternatively, a single robot may serve fewer bioreactors than any of the values listed herein, or a number of bioreactors falling within a range between any two of the values provided herein. In some embodiments, a single robot may serve a single bioreactor array. Optionally, multiple robots may serve a single bioreactor array. A single robot may serve multiple bioreactor arrays. In some instances, each bioreactor may have one or more dedicated robots. Optionally, multiple robots may be provided that may each serve multiple bioreactor arrays.

A bioreactor may have any dimension. For instance, a bioreactor may have a dimension (e.g., length, width, height, diagonal, or diameter) less than or equal to 1 cm, 3 cm, 5 cm, 10 cm, 15 cm, 20 cm, 25 cm, 30 cm, 35 cm, 40 cm, 45 cm, 50 cm, 60 cm, 70 cm, 80 cm, 90 cm, 1 m, 1.2 m, 1.5 m, or 2 m. A bioreactor may have a maximum dimension less than or equal to any of the values provided herein. A bioreactor may have a dimension greater than any of the values provided herein or falling within a range between any two of the values provided herein. A bioreactor may have a maximum dimension greater than any of the values provided herein, or falling within a range between any two of the values provided herein.

Optionally, a bioreactor may be a single-use bioreactor. The bioreactor may be disposable. Alternatively, the bioreactor can be reused. In some embodiments, one or more components of a bioreactor may be single-use/disposable. One or more components of a bioreactor may be reusable. A bioreactor may be removable from fermentation station. A bioreactor may be attached, detached, and/or reattached at the fermentation station. A bioreactor may be exchangeable with another bioreactor. In some embodiments, one or more receiving interfaces may be provided at a fermentation station, which may each be capable of receiving a bioreactor. In some embodiments, each receiving interface may be identical. A bioreactor may fit into any of the receiving interfaces at a fermentation station. Various bioreactors may be received at a receiving interface. In some instances, bioreactors with different settings or different configurations may be received at a receiving interface.

Each bioreactor (or ‘bay’) 310 may comprise one or more of the following components: media container 320, reactor vessel 330, sampling location 340, agitator 350, pump 360, control board 370, heater/cooler 380, imaging device 390, a condenser for exhaust gas, and/or a location for ‘run-time’ media additions.

A media container 320 may optionally be received from a seed train station of a workcell. The media container may comprise one or more media bottles. In another embodiment, a media container may be provided at a bioreactor and media may be provided to the media container with aid of a robot. The media container may be removable or detachable from the bioreactor. The media container may be single-use or reusable. The bioreactor may have a media container receiving region which may support and hold one or more media containers.

A reactor vessel 330 may be provided at a bioreactor. The reactor vessel may optionally be received from a seed train station of a workcell. The reactor vessel may comprise one or more containers, such as tubes, flasks, wells, plates, or any other type of container as described elsewhere herein. In another embodiment, a reactor vessel may be provided at a bioreactor and seed that has undergone seed train preparation may be added to the reactor vessel with aid of a robot. The reactor vessel may be removable or detachable from the bioreactor. The reactor vessel may be single-use or reusable. The bioreactor may have a reactor vessel receiving region which may support and hold one or more reactor vessels. A bioreaction may occur within the reactor vessel. For instance, a fermentation process may occur within a reactor vessel while coupled to the bioreactor. A reactor vessel may be formed from any material. For example, the reactor vessel may be formed from injection molded plastic, wood, iron, copper, glass, stainless steel, or other materials. The reactor vessel may be formed from a material that is substantively not corrosive, may be capable of tolerating high pressure, may be able to resist pH changes, may be able to tolerate steam sterilization, and/or may be free of toxins. Any description herein of experiments that may be conducted within a workcell may include a fermentation process that may occur in a bioreactor (e.g., reactor vessel of a bioreactor).

A sampling location 340 may be provided at a bioreactor. One or more sampling containers may be provided to the sampling location. In some embodiments, a sample within a sampling container may be collected from a reactor vessel 330. Sampling may occur at a single point during an experiment, or multiple points during an experiment. For example, multiple samples may be collected over time during an experiment. A separate sampling container may be used for each collection. In some embodiments, media may optionally be added from a media container to a reactor vessel. Media may be added from a media container to a reactor vessel at any point at the beginning of an experiment. The media may be added to a media container at a single point in time, or multiple points in time during an experiment. Media may or may not be added directly to a sampling container. In some embodiments, a sampling container may be stored at a sampling location until the sampling container is picked up and/or transported to a sampling handling station 230. Sampling containers at a sampling location may or may not undergo further fermentation.

In some embodiments, an agitator 350 may be provided at a bioreactor. For example, an agitator may provide magnetic agitation. In some instances, mechanical agitation, such as blades, may be employed. One or more impellers and/or baffles may be employed to aid in agitation. One or more agitation components (e.g., impeller) may be formed using 3D printing techniques. Agitation may be provided to contents of a reactor vessel. In some instances, agitation may be provided continuously. The level of agitation may be constant or may vary over time. In some instances, agitation may only be provided at selected time periods.

A bioreactor may comprise one or more pumps 360. The one or more pumps may control flow of a fluid, such as a liquid feed. A pump may remove liquid from the reactor vessel or to add acids and bases, antifoam reagents, and nutrients for continuous or batch cultures. The one or more pumps may control a flow of gas. For example, air or other gases may be added to the reactor vessel. In some embodiments, peristaltic pumps may be employed.

A bioreactor may have a control board 370. The control board may comprise one or more processors that may execute code, logic or instructions to perform one or more steps. The control board may generate instructions that may affect operation of the agitator, the pumps, heater/cooler, camera, sensors, and/or material handling. A control board may optionally control flow of one or more fluids. For instance, a control board may control flow of one or more gases into or out of a reactor vessel. A control board may control flow of one or more materials, such as media, to or from the reactor vessel.

The control board may generate instructions that may affect operation of one or more components of the bioreactor. The control board may generate instructions that may affect operation outside the bioreactor. The control board may receive instructions from other sources. For instance, the control board may receive instructions from other bioreactor control boards, from the cloud, from the robots, from any components within the system, or any components outside the system.

In some embodiments, the control board may be configured to generate instructions using a trained model. The trained model may process input sensor data and output a control signal/instruction. Alternatively or in addition to, the trained model may output an estimated state of the process or presence/level of foaming and an operational status of one or more components (e.g., pump, agitator, heater, cooler, etc) may be adjusted accordingly. In some cases, the trained model may process input data comprising one or more types of sensor data and output control instructions or one or more parameters to be adjusted for affecting the operation status of one or more components of the bioreactor. Details about the model for generating control instructions or operations are described later herein.

A bioreactor may comprise one or more memory storage units. The one or more memory storage units may comprise non-transitory computer readable media that may comprise code, logic, or instructions for executing one or more steps. The control board may execute one or more experiment protocols. A memory storage unit may store instructions for a particular experiment for the bioreactor.

A temperature control system 380 may be provided for a bioreactor. The temperature control system may comprise a heater and/or cooler that may control the temperature of the bioreactor. The temperature control system may control the temperature of the contents of a reactor vessel. In some embodiments, the temperature control system may be able to provide temperature control to the precision of at least 0.01 degrees C., 0.05 degrees C., 0.1 degrees C., 0.5 degrees C., 1 degree C., 2 degrees C., 3 degrees C., or 5 degrees C. Optionally, a temperature control system may comprise a water bath. A water bath may cool or heat a reactor vessel. In some instances, a temperature control system may comprise thermoelectric heating components. In some instances, Pelletier devices may be used.

A bioreactor may have an on-board camera 390. The on-board camera may be able to visualize the reaction taking place. For instance, the on-board camera may be able to capture images of the contents (e.g., culture medium) of the reactor vessel. In some instances, one or more on-board cameras may capture images of the sampling location and/or media containers. An on-board camera may be useful for detecting a stage of an experiment, and/or positioning of any physical components of the bioreactor. The on-board camera may capture image data that can be used to detect a foaming event/level for automated foaming control. Details about the foaming detection and control using image data are described later herein.

A bioreactor may optionally have a condenser for exhaust gas. The condenser may be provided for any type of gas that may be generated within the bioreactor. The condenser may be contained partially or completely within the bioreactor, or supported by the bioreactor.

In some instances, a bioreactor may comprise a location for ‘run-time’ media additions. In some instances, a media bottle may be provided that may be used for one-time media additions in the middle of an experiment. Any number of containers may be provided that may add materials, such as media, at any point during an experiment, which may occur on the bioreactor. The media additions may be partially or completely within the bioreactor, or supported by the bioreactor.

A bioreactor may comprise a housing or a substrate that may support one or more components of the bioreactor. A housing may partially or completely enclose one or more components of the bioreactor. In some instances, a bioreactor may comprise a head plate. Optionally, a bioreactor, housing, substrate, or head plate may be formed using 3D printing techniques.

A bioreactor may or may not have a local power source. For instance, a bioreactor may have an on-board energy storage system, such as a battery or capacitor. In some instances, a bioreactor need not have an on-board energy storage system and may receive power from another part of the workcell. In some instances, a fermentation station receiving interface may provide power to a bioreactor.

A robot may interact with a fermentation station 220. A robot may interact with one or more bioreactors 222. The bioreactors may have any of the qualities or characteristics described for FIG. 3. A robot may move one or more containers of the bioreactor. For instance a robot may move a sampling container to or from a sampling location. A robot may also move a sampling container from a sampling location to a sample handling station. A robot may interact with media containers. For instance, a robot may add or remove caps or other closures from the media containers. The robot may dispense liquids to or from the media bottles. In one example, a robot may dispense a media to a bioreactor media container from a seed train station 210 or from bulk media bottles 240. A robot may optionally dispense media from a bioreactor media container to a reactor vessel or other component of the bioreactor. Alternatively, the media may be automatically dispensed to a reactor vessel or other component of the bioreactor with aid of built-in tubing, piping, channels, or other techniques. In some instances, a robot may load a vessel into a bay. The robot may make any necessary liquid or fluid connections required. For example, a robot may put pumping lines into a peristaltic pump. The robot may also unload the vessel. The robot may make any necessary liquid or fluid disconnections when unloading the vessel. The robot may optionally put an unloaded vessel into a waste area. A robot may also optionally add liquids or other materials to a post-sterile addition system. A robot may add liquids to the bioreactor during an experiment. These may be referred to as runtime additions or post-sterile additions. Optionally, they are not continuous or semi-continuous feeds. Instead, they may happen once. For example, a robot may add anti-foam in response to foaming. A robot may add a certain molecule which may induce product formation. The robot may just add a certain media component that may not be needed at the beginning of an experiment but may be needed later on.

Each bioreactor may comprise one or more sensors. For example, one or more of the following sensors may be provided: temperature sensor, dissolved oxygen sensor, pH sensor, biomass concentration sensor (e.g., may measure optical density or other characteristics), UV Vis/Raman sensor, scales, and/or camera may be provided. Sensors may be reusable or may be single-use sensors. The sensors may be able to measure a quality of one or more components of the bioreactor, such as a reactor vessel, sampling location, media container, or any other component of the bioreactor. In one example, a bioreactor camera may be employed to visualize the contents (e.g., culture medium) of a reactor vessel or sampling container (e.g., color, tracking foam, tracking volume levels, etc.). The bioreactor camera may capture image data about the content in the bioreactor and the image data may be analyzed for detecting presence of bubbles/foaming or measuring a level of foaming. For example, the image may contain at least a portion of the bioreactor and/or a portion of the content in the bioreactor. The sensors of each bioreactor may be capable of operating independently of sensors on-board other bioreactors.

A sensor can be used to determine a foam level, emitted infrared light, emitted UV light, and emitted visible light from a bioreactor array. A sensor or device that is a part of a system or apparatus disclosed herein can measure for example, the mass inflow and volumetric outflow of air, nitrogen, oxygen, carbon dioxide, and methane.

A sample handling station 230 may permit sample preparation and/or analysis. A sample handling station may comprise one or more components for sample weighing 231, sample preparation 232, sample analysis 233, sample storage, and/or sample output. Any of these components or steps may be optional, provided in any order, or additional components or steps may be provided.

One or more samples may be weighed 231. The sample may be weighed within a sample container. The sample container may be provided from a fermentation station 220 or from the seed station. The sample may be provided from one or more bioreactors of a fermentation station. The sample may be provided within a container that was used at one or more bioreactors. The sample may be collected from a container that was used at one or more bioreactors and provided to a new container that is used for sample weighing. The sample and/or sample container may be provided with aid of a robot. The sample may be provided during any stage of experimentation. For instance, the sample may be provided to the sample handling station (e.g., for sample weighing) after completion of an experiment at a bioreactor. In some instances, the sample may be provided at the beginning, or any point during an experiment at the bioreactor. Multiple samples may be provided at multiple points in time.

Sample weighing may occur with aid of one or more sensors. For example, a scale may be employed to weigh the sample. The scale may have a high degree of accuracy and/or precision. In some instances, the scale may at least be accurate on the order of 0.00001, g, 0.00005 g, 0.0001 g, 0.0005 g, 0.001 g, 0.005 g, 0.01 g, 0.05 g, 0.1 g, 0.2 g, 0.5 g, 1 g, 2 g, 3 g, 5 g, 10 g, 15 g, 20 g, 30 g, or 50 g. Other techniques may be employed to detect an amount of sample (e.g., weight, volume, concentration, density, etc.).

An optical sensor, such as a barcode reader may also be employed to aid in sample measurement. For example, the optical sensor may read a symbol, such as a barcode, to detect the sample information, container information, source of the sample, experiment information relating to the sample, or any other information. The symbol may be used to track the sample, and information about the experiments conducted on the sample, the bioreactor used for the sample, or any other data of the sample may be added and/or accessible.

In some cases, a machine learning-based control mechanism may be employed to control the operational status of one or more components of the sample handling station 230. For instance, a trained model may process the input data such as image data and the output of the model may be control signals/commands to the sample handling station.

Sample preparation 232 may occur after sample weighing, concurrently with sample weighing, or subsequent to sample weighing. In some instances, sample preparation may occur with aid of one or more robots. Human intervention may not be required for sample preparation. A robot may operate equipment and perform liquid handling. The robot may be capable of interacting with and/or operating off-the-shelf equipment that does not requirement any modification to be used to by the robot. The robot may manipulate one or more sets of controls for the equipment (e.g., pressing buttons, flipping switches, turning dials, touching a touchscreen, opening/closing doors, etc.).

In some instances, sample preparation may comprise centrifugation. One or more centrifuges may be provided. A single centrifuge may accommodate a single sample at a time or multiple samples at a time. When multiple centrifuges are provided, they may be capable of operating independently of one another.

Sample preparation may include one or more separation processes. For instance, separation of cell pellet from supernatant may occur. Centrifugation may aid in separation, or other techniques or equipment may be used for separation.

Optionally, sample preparation may comprise additions of materials to the sample. For instance, liquid additions may be provided to lyse/stabilize cells or stabilize some analyte. Any type of materials may be added for sample lysing, stabilization, marking, reactions, or any other desired effect.

One or more sensors may be provided which may monitor activities of the various equipment and/or sample status. Optionally, data from the sensors may be used as feedback that may affect sample preparation.

Sample analysis 233 may occur after sample preparation, concurrently with sample preparation, or subsequent to sample preparation. In some instances, sample analysis may occur with aid of one or more robots. Human intervention may not be required for sample analysis. A robot may operate equipment for analysis. The robot may be capable of interacting with and/or operating off-the-shelf equipment that does not requirement any modification to be used to by the robot. The robot may manipulate one or more sets of controls for the equipment (e.g., pressing buttons, flipping switches, turning dials, touching a touchscreen, opening/closing doors, etc.). The robot may comprise a camera that may allow the robot to visually detect equipment, samples or other components of the system. For instance, a camera on the robot may allow the robot to read a screen or recognize controls of equipment, and may facilitate robot interaction with equipment. In some instances, the equipment for analysis may interface with equipment for sample handling. Alternatively, the equipment for analysis may operate independently of equipment for sample handling.

Sample analysis may optionally occur without requiring the aid of one or more robots. In some instances, equipment may be controlled without requiring robotic interaction. Additional methods to digitally control equipment may be employed. The workcell may communicate with the equipment to provide instructions for control or to read data collected by the equipment. In some instances, a device separate or external to the workcell (e.g., via the cloud) may communicate with the equipment to provide instructions for control or to read data collected by the equipment.

Equipment used for sample analysis may include, and is not limited to, equipment for biochemical analysis, ultraviolet (UV)/visible (Vis)/infrared (IR), high-performance liquid chromatography (HPLC)/gas chromatography (GC)/mass spectrometry (MS), Raman spectroscopy, DNA sequencing, RNA sequencing, protein quantification, cell-counting, cell imaging, microscope, or any other type of equipment. The sample may be analyzed for composition, properties, emissions, quantity, density, concentration, or any other quality or characteristic.

Data from the sample analysis may be further analyzed within the workcell or outside the workcell. Optionally, data generated from the sample analysis may be used as a control signal for bioreactor control.

In some instances, sample storage may be provided. Samples may be stored in a workcell for any length of time. For instance, samples may be stored in a manner that they may remain stable for at least 1 minute, 5 minutes, 10 minutes, 20 minutes, 30 minutes, 45 minutes, 1 hour, 2 hours, 3 hours, 4 hours, 6 hours, 12 hours, 24 hours, 36 hours, 48 hours, 72 hours, or longer. In some instances, the samples may be stored in cold storage. For instance, the samples may be stored in a cold container that may keep the samples below a desired temperature threshold.

Optionally, sample output 234 may be provided. Physical sample may be stored or provided outside the workcell. The physical sample storage may occur automatically without requiring human intervention. The sample may be provided in a manner that may allow the sample to be collected outside the workcell. In one example, cold storage of sample may be provided. A robot may aid in putting samples into cold storage. Samples may be broth or prepped in some manner (e.g., centrifugation or any other sample preparation step). In some instances, robots may aid in putting samples into desirable storage conditions (e.g., controlled temperature, controlled exposure to light or other radiation) and/or desired storage locations. A robot may put samples into liquid nitrogen to flash freeze them. A robot may aid in putting sample into a rack or box that a technician may be able to pick up. Optionally further handling or analysis of the sample output may occur outside the workcell.

In some embodiments, a workcell may permit cleaning up at the end of one or more experiments. This may include the removal of single use vessels. For example, the bioreactor vessels may be removed from the bioreactors when the experiment is concluded. The cleanup may occur automatically with aid of one or more robots. The cleanup may occur without requiring human aid or intervention. A robot may be capable of picking up vessels or other containers that are no longer needed and moving them to a different location. The removed containers may be sterilized, washed, or cleaned, for reuse. In some instances, this step may occur automatically without requiring human intervention. In some instances, the removed containers may be disposed or removed from the workcell.

One or more bulk media containers 240 may be provided within a workcell. The bulk media containers may comprise any type or number of containers (e.g., bottles, flasks, tubes, plates, wells, etc.). The bulk media containers may be entirely enclosed from the environment. Alternatively, one or more openings may be provided that may allow for exposure to the environment.

A bulk media container may be filled by a human operator. The containers may be filled inside or outside the workcell. Optionally, a robot may dispense media into the bulk media containers. A robot may or may not directly handle the bulk media container. In some instances, robots may be employed to handle media from the bulk media container and provide it to other containers within the workcell. In some embodiments, media may be directly metered and/or fed to other containers within the workcell. Media may be directly fed to one or more containers within a bioreactor.

Media containers may optionally be provided on scales 241 which may allow for precise metering of media. One or more pumps 242 may aid in dispensing the media. The pumps may dispense media from the bulk media containers. Alternatively or in addition, pumps may be employed to dispense media to the bulk media containers. In one example, pumps may be used to dispense media from the bulk media containers to one or more container at a seed train station 210, a fermentation station 220, and/or a sample handling station 230. Alternatively or in addition, robots may be employed to provide media from the bulk media containers to one or more containers at a seed train station 210, a fermentation station 220, and/or a sample handling station 230.

A workcell may comprise one or more robots 250. Any description here of a robot may apply to a robot arm, and vice versa. Any description of a robot may comprise one or more robotic components capable of actuation. A robot may comprise a robot arm. The robot arm may be a 6-axis robot arm. The robot arm may be capable of motion about 1 or more, two or more, three or more, four or more, five or more, or six or more axes of motion. The robot arm may comprise one or more, two or more, three or more, four or more, five or more, six or more, seven or more, eight or more, nine or more, or ten or more joints. The joints may comprise motors that may allow various support members to move relative to one another. The robot arm may comprise one or more, two or more, three or more, four or more, five or more, six or more, seven or more, eight or more, nine or more, or ten or more support members. In one example, a first support member may bear weight of an end effector. A second support member may bear weight of the first support member and/or the end effector, and so forth. The motors may allow rotation of one or more support members relative to one another. One or more sliding mechanism may be provided that may allow lateral displacement. One or more telescoping components for support members may or may not be provided. The robot arm may have a free range of motion that may match or exceed the range of motion of a human arm. Ball and socket joints may or may not be employed by the robot arm. A robot arm can move an inoculant from an automated seed system to the reactor.

The robot may comprise a robot carriage 251. In some embodiments, a robot arm may be supported on a robot carriage. The robot carriage may bear weight of the robot arm. The robot carriage may support the robot arm. The robot carriage may support a robot arm on a top surface of the robot carriage, a bottom surface of the robot carriage, and/or a side surface of the robot carriage. A robot carriage may support a single robot arm or multiple robot arms. One or more robot arms may be affixed to the carriage or may be movable relative to the robot carriage at the location where the robot is supported by the robot carriage. Robot arms supported by the robot carriage may have the same characteristics or may have one or more differing characteristics (e.g., size, number/type/direction of joints, number/type/characteristics of support members, end effectors, materials, etc.).

The robot carriage may be capable of motion. The robot carriage may move relative to the rest of the workcell. The robot carriage may move relative to one or more bioreactors. The robot carriage may move relative to equipment used for seed train preparation, and/or sample handling. The robot carriage may be supported be a support mount, such as a linear rail 252. The robot carriage may move in a translational manner along the support mount. For instance, the robot carriage may move laterally and/or vertically along a support mount. The support mount may comprise one or more straight lines, curves, and/or corners. The support mount may be formed from a single track or may comprise multiple tracks that the robot carriage may follow. A support mount may be elevated. The support mount may be supported by a workcell floor, wall, and/or ceiling. In some instances, a location of a robot may be measure and/or monitored with aid of the support mount. In some instances the support mount may have a known location and the location of the robot carriage relative to the support mount may be determined. In some embodiments, one or more motors and/or sensors may be provided on a support mount, such as a linear rail, to effect movement of the robot carriage. Optionally, one or more motors and/or sensors may be provided on a robot carriage to effect movement of the robot carriage.

In some instances, the robot carriage may be capable of movement without being restricted to a track or rail. The robot carriage may move autonomously or semi-autonomously. In some instances, the robot carriage may move across a surface. For example, one or more sets of wheels, legs, arms, treads, gliders, or other components may be used to propel a robot carriage. A robot carriage may drive along a floor of a workcell. A robot may be supported by a quadcopter or other type of flying vehicle. A robot may be capable of flight within a workcell.

The robot carriage may optionally bear weight of one or more bulk media containers 240. The robot carriage may or may not support one or more bulk media containers. One or more bulk media containers may move with the robot carriage. For instance, if a robot carriage navigates a rail, the bulk media containers may move along with the robot carriage along the rail.

A location and/or position of the robot may be monitored. In some embodiments, one or more sensors on a robot arm, robot carriage, support mount, or other portion of the workcell may be used to determine the location of the robot within the workcell and position of one or more components of the robot. This may be useful when the robot needs to execute precise motions in interacting with various components of the workcell. In one examples, servomotors may be employed that may be useful for determining position, speed, or acceleration of the robot, or one or more components of the robot.

In one example, a robot may comprise an end effector. For instance, one or more end effectors may be positioned at an end of a robot arm. In some instances, end effectors may be provided at other locations along a robot arm. An end effector may interact with one or more other component of a workcell. For instance, an end effector may manipulate or interact with one or more containers or equipment. An end effector may be used to lift and/or transport a container. An end effector may be used to rotate or flip a container. An end effector may be used to interact with equipment (e.g., press a button, flip a switch, turn a dial, open/close a door, touch a touchscreen, etc.).

Various types of end effectors may be employed. In one example, an end effector may comprise a gripper. A gripper may grasp one or more objects. A gripper may comprise two or more ‘fingers’ that may be capable of movement relative to one another. A gripper may be moved relative to the rest of the arm and allow an object held by the gripper to move rotationally and/or translationally.

In some embodiments, an end effector may utilize magnets, vacuum suction, fasteners, cutters, sensors (e.g., cameras, barcode readers, microphones, etc.), emitters (e.g., light, sound), or other components to sense and/or interact with other components of the workcell. In one example, an end effector may comprise a pipettor. In another example, an end effector may comprise an optical detector, such as a camera or barcode reader. Different types of end effectors may be provided. In some instances, multiple of the same type of end effectors may be provided. They may have the same dimensions or other characteristics, or different dimensions or other characteristics.

An end effector may move in any direction. For instance, an end effector supported by a robotic arm, may translate along one or more, two or more, or three or more axes, or may rotate about one or more, two or more, or three or more axes. An end effector may rotate about a roll axis, pitch axis, and/or yaw axis.

In some embodiments, multiple types of end effectors may be utilized by a robot. The end effectors may be swappable 253. For example, a first end effector may be removed from a robot arm. A second end effector may then be attached from the robot arm. The first end effector and the second end effector may be of the same type or different types. The first end effector and the second end effectors may have the same characteristics or may have at least one characteristic that is different. In some instances, a robot arm may utilize a single end effector at a time. Alternatively, a robot arm may be capable of utilizing multiple end effectors at a time. A workcell may have one or more locations where end effectors that are not being used by the robot are stored. The robot may drop off and/or pick up new end effectors as needed. The robot may swap end effectors according to need. In some embodiments, a workcell may comprise multiple robots. The multiple robots may share the same pool of end effectors. Alternatively, each robot may have its own set of end effectors that it may access.

A work cell may comprise one or more sensors for environmental monitoring 260. The environmental monitoring sensors may be capable of detecting one or more conditions within a workcell. The sensors may detect conditions at particular regions or stations of the workcell, or the workcell overall. The sensors may include, and are not limited to, temperature sensors, pressure sensors (e.g., air pressure sensor), gas detectors (e.g., detecting ambient O₂, CO₂, or other gases), motion sensors, particulate sensors, microphones, optical sensors, or other sensors. The sensors may be useful for detecting whether the environment is sterile or whether contamination has occurred. The sensors may be useful for detecting an error condition. The sensors may be useful for detecting whether the environment is conducive to various experimental parameters for the bioreactors.

A work cell may comprise one or more cameras 270. A camera may monitor activity within the workcell. In some instances, data collected by a camera may be automatically analyzed with aid of one or more processors. In some instances, a human may view images captured by a camera in real-time or at a later time. In some instances, cameras may be useful for monitoring completion and timing of tasks. This may be useful for determining when human tasks are required, for example, when doors open. For instance, cameras may be useful for determining when experiments are complete.

A single camera may be provided within the workcell. Alternatively, multiple cameras may be provided within a workcell. Different cameras may have different fields of view. For instance, different cameras may be used to capture images of different regions of the workcell. The cameras may have a fixed position relative to the rest of the workcell. Alternatively, the cameras may be movable relative to the rest of the workcell. In some examples, the cameras may rotate about one, two, three or more axes. The motion of the camera may optionally be remotely controlled from outside the workcell.

### Foam Detection and Control System

In one aspect, the present disclosure provides systems and methods for foam detection and prediction during fermentation. The provided foam detection and prediction mechanism may be capable of detecting presence of foam and/or level of foaming based on image data. In some embodiments, the foam detection and prediction mechanism may be applied to a foam control system such that one or more operational status of a bioreactor (e.g., airflow to redirect from bottom feed of the sparger to feeding into the headspace of the bioreactor, pressure, temperature and agitation speed, etc) and/or conditions of a fermentation process (e.g., temperature, pressure, mixing or pH, etc) may be automatically adjusted based on the detection result.

In some embodiments, the image data may contain an image of at least a portion of the reactor vessel. The image data may comprise an image of the content in the bioreactor. The reactor vessel may be composed of an optically transmissive material, such as one or more of a translucent material, a transparent material, a semi-transparent material, or a semi-translucent material such that content in the reactor vessel can be visible.

The image data may be processed to detect presence of bubbles/foam or a level of foaming. In some cases, the image data may be processed by a trained model comprising a binary classifier to determine presence of foam. Binary classifiers may comprise supervised machine learning models that help the system in making accurate predictions. These classifiers may first be trained by users (e.g., human experts) on a set of training data and may later be used for prediction in real time. The system may comprise one, two, three, four or more different types of classifiers. The image data may be processed by the binary classifiers to determine whether the bubbles or foaming is in presence.

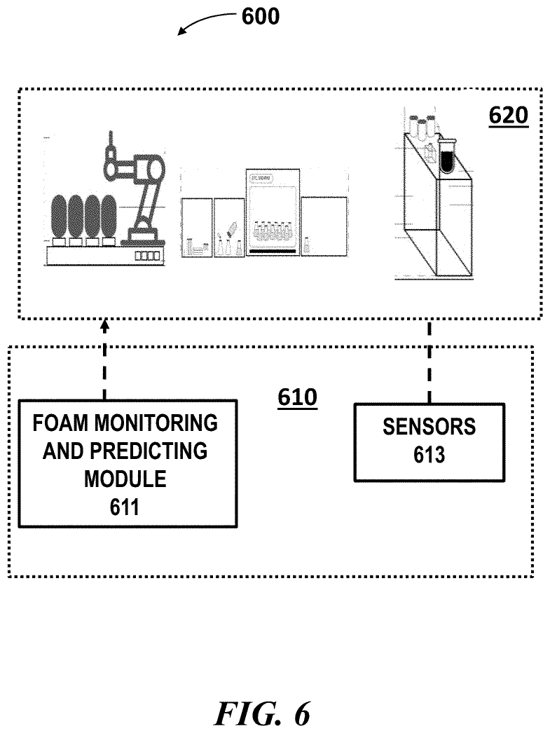

In some cases, the classifier may be trained to output a level of foaming or an amount of foaming (e.g., volume of foam, mean average size of bubbles, density of bubbles, etc). FIG. 6 shows examples of different levels of foaming (e.g., conditions) predicted by a trained model. In some cases, the detected level of foaming may be used to determine an amount of antifoam agent to be added to the reactor vessel.

In some cases, the classifier may be trained to predict the occurrence of foaming. For instance, the classifier may process time-series data (e.g., image data stream) proceeding the presence of foaming to predict an impeding foaming event. In some cases, the image data may be pre-processed prior to being processed by the classifier. For instance, feature extraction may be applied to extract various features from the raw image data and the extracted features may be processed by the classifier. For example, the classifier may be capable of predicting one or more pre-foaming statuses or the conditions indicating foaming is likely to happen. In response to such detection, operational status of a bioreactor (e.g., airflow to redirect from bottom feed of the sparger to feeding into the headspace of the bioreactor, pressure, temperature and agitation speed, etc) and/or conditions of a fermentation process (e.g., temperature, pressure, mixing or pH, etc) may be automatically adjusted to prevent foaming from occurring.

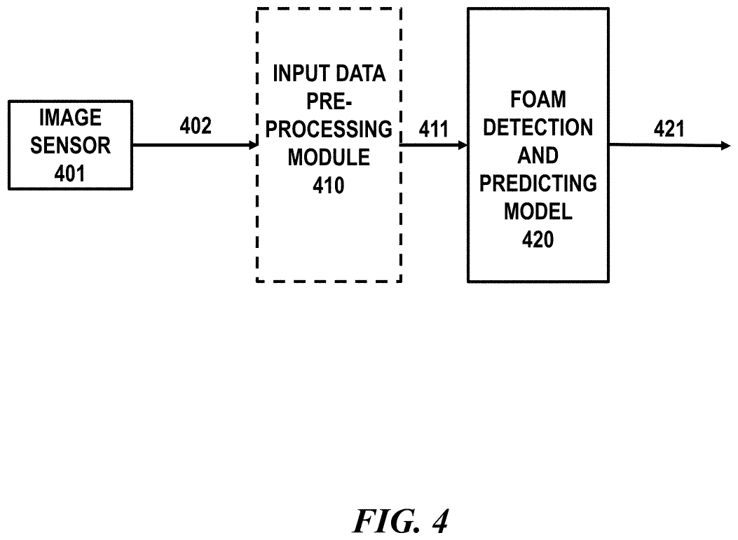

FIG. 4 shows an exemplary process of processing image data using a trained model for foam detection and prediction. The image data 402 may be captured by an image sensor 401. The image data may optionally be pre-processed by an input data pre-processing module 410 and the pre-processed data 411 may be further processed by a foam detection and predicting model 420 to generate an output result 421.

The image sensor 401 can be the same as the image sensor equipped with the bioreactor and/or the fermentation system. For example, the image sensor can be the on-board camera that is used to visualize the reaction taking place. As described above, the on-board camera may be able to capture images of the contents of the reactor vessel. In some instances, one or more on-board cameras may capture images of the media containers. An on-board camera may be useful for detecting a stage of an experiment, and/or positioning of any physical components of the bioreactor.

The captured image data 402 may be color images (RGB images) or greyscale images. The image data may be time-series data. In some cases, the image data may be pre-processed to generate a feature vector. For example, low-pass filtering or normalization may be applied to the input data such that the processed data has zero mean and unity variance.

In some cases, the image data 402 may be pre-processed by an input data pre-processing module 410. For instance, one or more techniques such as normalization or filtering may be applied to the image data to improve the quality of the image data before being processed by the foam detection and predicting model. Non-limiting examples of these filters can be convolutional filters (for example Roberts edge enhancing filter, Gaussian smoothing filter, Gaussian sharpening filter, Sobel edge detection filter, etc.) or morphological filters (for example erosion, dilation, segmentation filters, etc.) or various other filters. These filters may enhance image parameters such as SNR or resolution. Other methods for improving image quality such as methods making use of local patch statistics, prior temporal information, denoising methods, such as the HYPR processing, non-local mean denoising, guided image filtering, entropy or mutual information based methods, segmentation based methods or gradient based methods can also be applied.

In some embodiments, the input data pre-processing module 410 may be configured to pre-process the image data to extract features. The input data pre-processing module 410 may employ supervised learning, semi-supervised learning or un-supervised learning techniques to extract features from the raw image data. For example, the input data pre-processing module 410 may comprise an autoencoder for feature extraction. During the feature extraction operation, the autoencoder may be used to learn a representation of the input data for dimensionality reduction or feature learning. The autoencoder can have any suitable architecture such as a classical neural network model (e.g., sparse autoencoder, denoising autoencoder, contractive autoencoder) or variational autoencoder (e.g., Generative Adversarial Networks). In some embodiments, a sparse autoencoder with an RNN (recurrent neural network) architecture, such as LSTM (long-short-term memory) network, may be trained to regenerate the inputs for dimensionality reduction. For example, an encoder-decoder LSTM model with encoder and decoder layers may be used to recreate a low-dimensional representation of the input data to the following model training despite a latent/hidden layer. For example, supervised features may be extracted automatically from time series data (e.g., sequence of image data). The supervised features can be of any type. For instance, the supervised features may represent FFT amplitude or any suitable supervised feature of time-series data such as skew, kurtosis, power, energy, entropy, RMS, mean, variance, standard deviation, signal magnitude and the like. In some cases, the autoencoder may extract features that best characterize temporal data. Alternatively or in addition to, the autoencoder may extract features that are useful for event prediction. The feature extraction operation though is described as operations performed by the input data pre-processing module, such operations may also be perform by the foam detection and prediction model. For instance, the autoencoder for feature extraction may be a part of the foam detection and prediction model.

The processed data 411 may comprise input feature vectors to be supplied to the foam detection and prediction model 420. The foam detection and prediction model 420 may comprise a trained model. In some embodiments, the trained model may comprise one or more classifiers for generating a foam detection result, foam prediction result or direct control commands/signals. The classifiers can be of any suitable type, including but not limited to, KNN (k-nearest neighbor), support vector machine (SVM), a naïve Bayes classification, a random forest, decision tree models, convolutional neural network (CNN), feedforward neural network, radial basis function network, recurrent neural network (RNN), deep residual learning network and the like.

In some cases, the model network for training may comprise an autoencoder and a classifier system. For example, an autoencoder may be used for feature extraction operation by learninig a representation of the input data for dimensionality reduction or feature learning. The autoencoder can have any suitable architecture as described above.

One or more components of the foam detection and prediction model (e.g., classifiers, autoencoder) may be trained using supervised learning techniques, semi-supervised learning or un-supervised learning techniques. For example, the training method may involve pre-training one or more components of the predictive model, an adaptation stage that involves training the predictive model to adapt to a fermentation system in which the pre-trained model is implemented, and an optimization stage that involves further continual tuning of the predictive model or a component of the predictive model (e.g., classifier) to adapt to changes in the implementation environment over time (e.g., changes in the fermentation system, model performance, organism/experiment-specific data, etc).

In some cases, the foam detection and prediction model may undergo supervised learning that requires labeled datasets. In some cases, labeled datasets (e.g., image data, reference/ground truth data) may be retrieved from a database, external data sources, or provided by one or more users or fermentation systems. In some cases, the labeled data may be provided by experts or skilled person (e.g., engineers, scientists) or calculated based on existing/empirical data using a known formula. For example, the reference data or ground-truth data may be a binary result (e.g., presence of bubble/foam or not) which can be provided by one or more users. In some cases, the reference data or ground-truth data may comprise information about the addition of an amount of one or more antifoam agents. For example, the amount of antifoam agents (e.g., volume) or one or more parameters about adding the antifoam agents (e.g., flow, duration, time at which to remove the antifoam agents, etc) may be provided by an engineer or skilled person, or calculated based on empirical data (e.g., data collected from one or more fermentation systems) and a known formula/model.

The foam detection and prediction model may be capable of predicting an impeding foaming event or predicting the occurrence of foaming. The foam detection and prediction model may predict a foaming event with a pre-determined number of data points before the occurrence of a foaming event, and early intervention may be performed such that the amount of antifoaming agents may be reduced. The foam detection and prediction model may comprise a trained classifier that may process time-series data collected preceding the occurrence of foaming and output detection result indicating foam will occur. In the case of foaming prediction, the reference data or ground-truth data may comprise control signals or control commands to adjust operational status of one or more components of the bioreactor (e.g., airflow to redirect from bottom feed a the sparger to feeding into the headspace of the bioreactor, pumping or releasing of gas, pressure, temperature and agitation speed, etc) and/or conditions of a fermentation process (e.g., temperature, pressure, mixing or pH, etc).

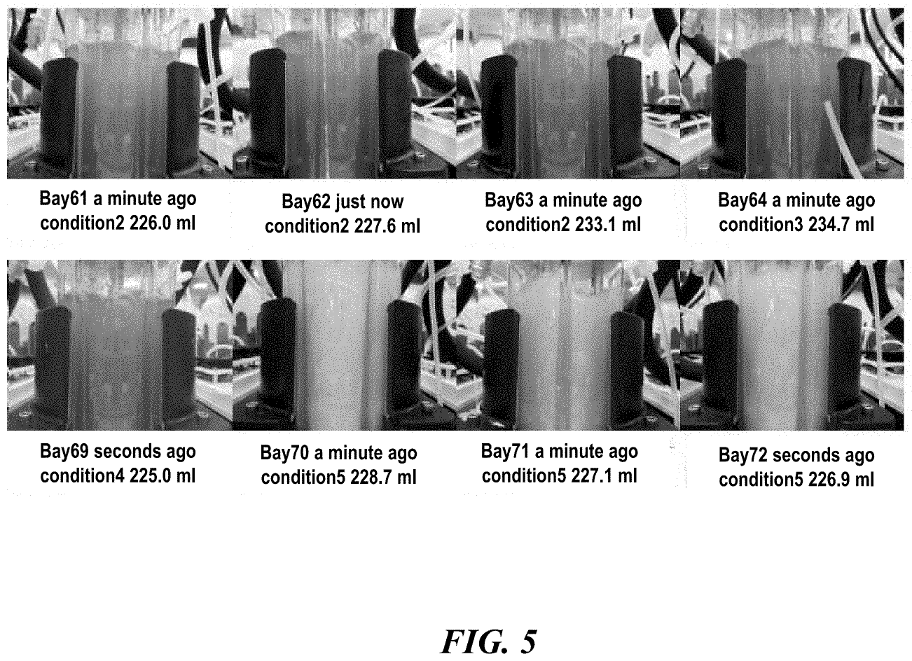

FIG. 5 shows examples of images captured by an imaging device for foaming prediction. The images (e.g., time-series data) may be captured every 1 second, 10 seconds, 20 seconds, 30 seconds, 1 minutes, 2 minutes, 5 minutes, 10 minutes, 1 hour and the like. The images may be processed in real-time for automated foaming control. The aforementioned foam detection and prediction system and method may be applied to a fermentation system. In the illustrated examples, the image may be processed to predict a condition related to foaming/fermentation (e.g., condition 2, condition 3, condition 5). For example, the different conditions may indicate different levels or types of foaming.

In some cases, an alert or notification may be generated when an impeding foaming is predicted. A notification about the predicted occurrence of foaming event may be generated and provided to a user. Such output result may be delivered to the user via any suitable approach or be in any suitable form, such as audio, visual, or tactile feedback. The notification or output result may be delivered through a user device. The notification may comprise an alert that can be delivered in any suitable forms (e.g., audio, visual alert in a GUI, webhooks that can be integrated into other applications, etc) or via any suitable communication channels (e.g., email, Slack, SMS). In some cases, the output result may also be delivered to any entities that are monitoring the experiment run or the fermentation process.

The collected real-time image data may also be used for continual training of a predictive model. In some cases, the predictive model may be further optimized to better adapt to the physical fermentation system or experiment-specific data. For example, the autoencoder and/or classifier may be further tuned as new image data and/or labeled data are collected. This continual learning approach may beneficially improve the model performance over time and improve the model's adaptability to changes in the fermentation system, experiment or organism characteristics or other variables over time.

FIG. 6 schematically illustrates a fermentation system 600 comprising a foam detection and prediction sub-system 610. The foam detection and prediction sub-system 610 may be a standalone system or a component of the fermentation system. The foam detection and prediction system 610 may comprise one or more optical imaging sensors 613. The one or more optical imaging sensors may be used to detect presence of foaming, measuring a degree/level of foaming or predicting the occurrence of foaming. The one or more optical imaging sensors may be configured to collect image data. The captured image data may be transmitted to the foam monitoring and predicting module 611 for processing. The foam monitoring and predicting module may implement a foam monitoring and predicting model as described above.

In some cases, the one or more imaging sensors 613 may be packaged within an imaging device such as a camera. Examples of imaging devices may include a camera, a video camera, or any device having the ability to capture optical signals. The imaging device may be configured to acquire and/or transmit one or more images of the reactor vessel or contents in the reactor vessel within the imaging device's field of view. The imaging device may have a field of view of at least 80 degrees, 90 degrees, 100 degrees, 110 degrees, 120 degrees, 130 degrees, 140 degrees, 150 degrees, 160 degrees, or 170 degrees. In some instances, the field of view may be fixed. In some instances, the field of view may be adjustable. The imaging device may be mounted to any suitable location as long as at least a portion of the vessel is captured in the image data. The imaging device may be a 2D camera or a 3D camera.

The imaging sensors may be configured to generate image data in response to various wavelengths of light. For example, the imaging sensors may be configured to collect images in the ultraviolet, visible, near-infrared, or infrared regions of the electromagnetic spectrum. A variety of imaging sensors may be employed for capturing image data such as complementary metal oxide semiconductor (COMS) or charge-coupled device (CCD) sensors. In some cases, the output of the imaging sensor may be image data (digital signals) that may be processed by a camera circuit or processors of the camera. In some cases, the imaging sensor may comprise an array of optical sensors.

The imaging sensor may capture an image frame or a sequence of image frames at a specific image resolution. The image frame resolution may be defined by the number of pixels in a frame. For example, the image resolution may be greater than or equal to about 352×420 pixels, 480×320 pixels, 720×480 pixels, 1280×720 pixels, 1440×1080 pixels, 1920×1080 pixels, 2048×1080 pixels, 3840×2160 pixels, 4096×2160 pixels, 7680×4320 pixels, or 15360×8640 pixels. The imaging device may capture color images (RGB images), greyscale image, and the like.

The imaging sensor may capture a sequence of image frames at a specific capture rate. In some embodiments, the sequence of images may be captured at standard video frame rates such as about 24p, 25p, 30p, 48p, 50p, 60p, 72p, 90p, 100p, 120p, 300p, 50i, or 60i. In some embodiments, the sequence of images may be captured at a rate less than or equal to about one image every 0.0001 seconds, 0.0002 seconds, 0.0005 seconds, 0.001 seconds, 0.002 seconds, 0.005 seconds, 0.01 seconds, 0.02 seconds, 0.05 seconds. 0.1 seconds, 0.2 seconds, 0.5 seconds, 1 second, 2 seconds, 5 seconds, 10 seconds, 20 seconds, 30 seconds, 1 minutes, 2 minutes, 5 minutes, 10 minutes, 1 hour and the like for viewing the status of the experiment in real-time or for monitoring and detecting a state of the fermentation process. In some embodiments, the capture rate may change depending on user input and/or external conditions (e.g., illumination brightness).

In some cases, the imaging device may include a zoom lens for which the focal length or angle of view can be varied. The imaging device may provide optical zoom by adjusting the focal length of the zoom lens.

In some cases, the imaging device may be in communication with the foam monitoring and predicting module 611. Image data collected by the imaging device may be transmitted to the foam monitoring and predicting module. The image data may be transmitted via a wired or wireless connection. In some cases, the imaging device may be in communication with other devices. For instance, the imaging device may transmit image data to a display such that one or more images or video streams may be displayed to a user or operator monitoring the fermentation process.

The imaging device may be in communication with the control module of the bioreactor or fermentation system 620. For example, the imaging device may receive control signals from a control module of the bioreactor for controlling the operation of the camera (e.g., taking still or video images, zooming in or out, turning on or off, switching imaging modes, changing image resolution, changing focus, changing depth of field, changing exposure time, changing viewing angle or field of view, etc.).

### Status Estimation System

In one aspect of the present disclosure, system and method are provided for estimating or predicting status/state of a fermentation process. The predictive system may be capable of estimating a state of the fermentation process based one multiple types of sensor data such that the operational status of one or more components of a bioreactor (e.g., airflow to redirect from bottom feed a the sparger to feeding into the headspace of the bioreactor, pressure, temperature and agitation speed, etc) and/or conditions of a fermentation process (e.g., temperature, pressure, mixing or pH, etc) may be automatically adjusted based on the estimation result.

In some embodiments, the input data to the predictive system may comprise one or more types of sensor data. The one or more types of sensor data may comprise data captured by an imaging device, data captured by temperature sensor, pH sensor, optical density (OD) and various other sensors provided with the bioreactor or fermentation system as described above.

The input data may be processed by one or more trained classifiers. In some cases, these classifiers may first be trained by human experts on a set of training data and may later be used for prediction in real time. The system may comprise one, two, three, four or more different types of classifiers. In some cases, the input data may be pre-processed prior to being processed by the classifier. For instance, feature extraction may be applied to extract various features from the raw sensor data and the extracted features may be processed by the classifier. In some cases, the features may be parameters of the fermentation process, including but not limited to, pressure, pH value, temperature, presence of foam and the like. In some cases, at least one of these parameters may be derived from one or more types of sensor data.

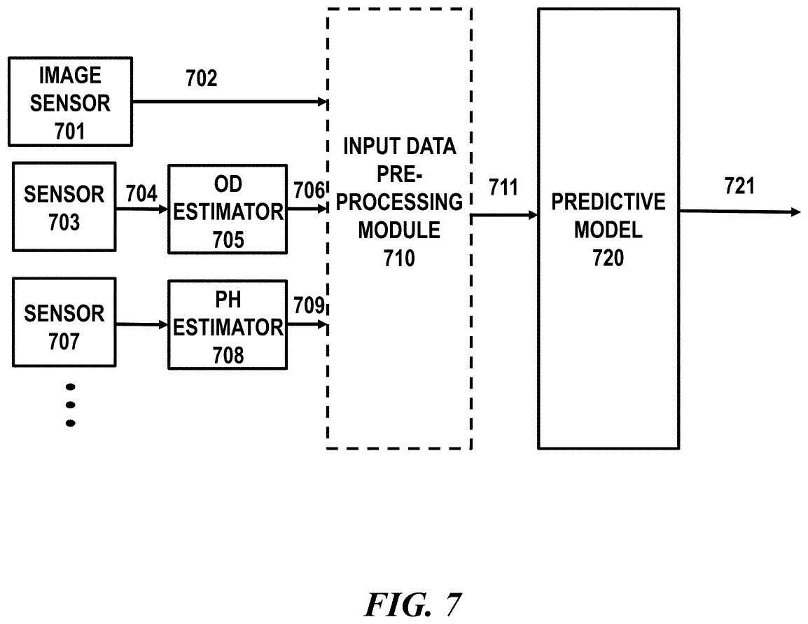

FIG. 7 schematically shows block diagrams of processing sensor data using a trained model for state estimation. The input data may comprise raw sensor data (e.g., image data) and/or data derived from one or more types of sensor data. The input data may optionally be pre-processed by an input data pre-processing module 710. The processed data 711 may be further processed by a predictive model 720 to generate an output result 721.

The input data may comprise raw sensor data such as image data 702 captured by an image sensor 701. In some cases, the input data may comprise data derived from one or more types of sensor data. The derived data may be a key parameter for a fermentation process. In some cases, the key parameters (e.g., pH, OD, etc) may be selected for characterizing different stages or states of a fermentation process. In some cases, the key parameters may not be directly measurable due to the limited reliability of sensors. The one or more key parameters may be estimated or derived based on sensor data. The key parameter may be estimated based on one or more other parameters/metrics or different types of sensor data. For instance, parameters such as pH 709, dissolved oxygen tension (DOT), optical density (OD) 706 and temperature may be derived from one or more types of measurements or metrics.

As an example, the OD parameter 706 or pH parameter 709 may be estimated based on one or more measurements captured by one or more sensors 703, 707. The one or more sensors 703, 707 may be used to generate measurements of different metrics that may be fused and processed by an OD estimator 705 and/or pH estimator 708 for estimating OD 706 and pH 709. For instance, metrics such as offline carbon measurements and respiration data from a reactor may be combined and analyzed by the OD estimator 705 to estimate the OD 706. The one or more sensors 703, 707 may be shared and the collected sensor data may be used for estimating different parameters (e.g., pH, DOT, OD, temperature, etc). For example, image data may be used to augment the input data for estimating OD.

The direct measurements of metrics for estimating a key parameter may be pre-selected. In some cases, the estimator such as OD estimator 705 or pH estimator 708 may comprise a model for estimating a key parameter. The input to an estimator may comprise one or more types of sensor data, one or more metrics that may be pre-determined based on the model. In some cases, the estimator may employ sensor fusion techniques or other suitable techniques to improve the accuracy or reliability of the estimation. In some cases, the model may be a model trained using machine learning techniques as described elsewhere herein.

The image sensor 701 can be the same as the image sensor as described elsewhere herein. For example, the image sensor can be an on-board camera that is used to visualize the reaction taking place. As described above, the on-board camera may be able to capture images of the contents of the reactor vessel. In some instances, one or more on-board cameras may capture images of the sampling location and/or media containers. An on-board camera may be useful for detecting a stage of an experiment, and/or positioning of any physical components of the bioreactor.

The input data (e.g., image data 702, pH 709, OD 706) may be pre-processed by an input data pre-processing module 710. In some embodiments, data processing techniques such as data normalization, labeling data with metadata, data annotation, data enrichment, tagging, data alignment, data segmentation, and various others may be performed by the input data pre-processing module 710. For example, data captured by different sensors (e.g., sensors may capture data at different frequency) may be aligned. For instance, data captured by camera, temperature sensor, OD sensor and the like may be aligned with respect to time. In some cases, data alignment may be performed automatically. Alternatively or in addition to, a user may specify the data collected from which sensors or sources are to be aligned and/or the time window during which data is to be aligned. As an example, the result data may be time-series data aligned with respect to time.

The input data pre-processing module 710 may perform other data processing as described elsewhere herein. For example, normalization or filtering may be applied to the image data to improve the quality of the image data. Non-limiting examples of these filters can be convolutional filters (for example Roberts edge enhancing filter, Gaussian smoothing filter, Gaussian sharpening filter, Sobel edge detection filter, etc.) or morphological filters (for example erosion, dilation, segmentation filters, etc.) or various other filters. These filters may enhance image parameters such as SNR or resolution.

In some embodiments, the input data pre-processing module 710 may be configured to pre-process the input data by employing supervised learning, semi-supervised learning or un-supervised learning techniques. For example, the input data pre-processing module 710 may comprise an autoencoder for feature extraction. During the feature extraction operation, the autoencoder may be used to learn a representation of the input data (e.g., image data 702, pH 709, OD 706) for dimensionality reduction or feature learning. The autoencoder can have any suitable architecture such as a classical neural network model (e.g., sparse autoencoder, denoising autoencoder, contractive autoencoder) or variational autoencoder (e.g., Generative Adversarial Networks). In some embodiments, a sparse autoencoder with an RNN (recurrent neural network) architecture, such as LSTM (long-short-term memory) network, may be trained to regenerate the inputs for dimensionality reduction. For example, an encoder-decoder LSTM model with encoder and decoder layers may be used to recreate a low-dimensional representation of the input data to the following model training despite a latent/hidden layer.

The processed data 711 may comprise input feature vectors to be fed to the predictive model 720. The predictive model 720 may be a machine learning algorithm trained model. In some embodiments, the trained model may comprise one or more trained classifiers for generating a state estimation result or control commands/signals to one or more components of the fermentation station. The classifiers can be of any suitable type, including but not limited to, KNN (k-nearest neighbor), support vector machine (SVM), a naive Bayes classification, a random forest, decision tree models, convolutional neural network (CNN), feedforward neural network, radial basis function network, recurrent neural network (RNN), deep residual learning network and the like.

One or more components of the predictive model (e.g., classifiers, autoencoder) may be trained using supervised learning techniques, semi-supervised learning or un-supervised learning techniques. For example, the training method may involve a pre-training stage for training one or more components of the predictive model, an adaptation stage that involves training the predictive model to adapt to a fermentation system in which the pre-trained model is implemented, and an optimization stage that involves further continual tuning of the predictive model or a component of the predictive model (e.g., classifier) to adapt to changes in the implementation environment over time (e.g., changes in the fermentation system, model performance, organism/experiment-specific data, etc).

In some cases, the predictive model may undergo supervised learning that requires labeled datasets. In some cases, labeled datasets or training datasets (e.g., sensor data, reference/ground truth data) may be retrieved from a database, external data sources, or provided by one or more users or fermentation systems. In some cases, the labeled data may be provided by experts or skilled person (e.g., engineers, scientists) or calculated based on existing/empirical data using a known formula. For example, the reference data or ground-truth data may be a binary result (e.g., presence of bubble/foam or not) or level of foaming (e.g., different foaming conditions) which can be provided by one or more users. In some cases, the reference data or ground-truth data may comprise estimated stage of the fermentation process. In some cases, the reference data or ground-truth data may comprise control commands for operating one or more components of the fermentation system (e.g., pump, agitator, heater, cooler, etc).

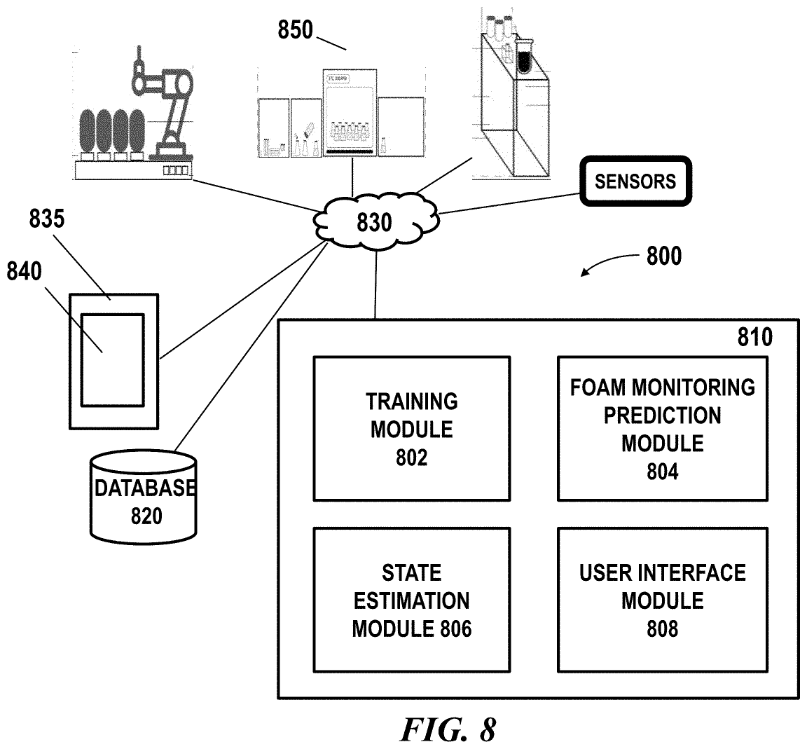

FIG. 8 schematically illustrates a monitoring system 800 for monitoring and controlling a fermentation process. The monitoring system 800 may comprise multiple components, including but not limited to, a training module 802, a foam monitoring and predicting module 804, a state estimation module 806 and a user interface module 808.

The training module 802 may be configured to obtain and manage training datasets. The training module 802 may be configured to train one or more models for detecting and predicting foaming, foam control, for estimating fermentation state or for controlling fermentation process as described elsewhere herein. For example, the training module may employ supervised training, unsupervised training or semi-supervised training techniques for training the model. The training module may implement the machine learning methods as described elsewhere herein. The training module may train a model off-line. Alternatively or additionally, the training module may use real-time data as feedback to refine the model for improvement or continual training. In some cases, the training module may implement the method described in FIG. 4 or FIG. 7 to further improve the performance of the system by using sensor data from the fermentation system 850.

The foam monitoring and predicting module 804 may be configured to monitor and control foaming using a trained model obtained from the training module. The foam monitoring and predicting module may implement the trained model for making inferences, i.e., detecting presence/level of foaming, predicting foaming, or generating control signals. The foam monitoring and predicting module can be the same as the foam monitoring and predicting module as described in FIG. 6. In some cases, the foam monitoring and predicting module may implement the method as described in FIG. 4 to further improve the performance of prediction.

The state estimation module 806 may be configured to monitor fermentation states and control one or more parameters/conditions of fermentation using a trained model obtained from the training module. The state estimation module 806 may implement the trained model for making inferences, i.e., estimating a fermentation state or generating control signals. In some cases, the state estimation module may implement the method as described in FIG. 7 to further improve the performance of prediction. The state estimation module 806 can be the same as the foam monitoring and predicting module as described in FIG. 7.

The user interface (UI) module 808 may be configured for representing and delivering fermentation run analytics (e.g., sensor data, fermentation process), or real-time sensor data (e.g., video). The UI may include a UI for representing real-time predictions generated by the state estimation module 806 or foam monitoring and predicting module 804 to the user and receiving user input from a user (e.g., through user device).

The user interface (UI) module 808 may generate one or more graphical user interfaces (GUIs). The GUIs may be rendered on a display screen on a user device. A GUI is a type of interface that allows users to interact with electronic devices through graphical icons and visual indicators such as secondary notation, as opposed to text-based interfaces, typed command labels or text navigation. The actions in a GUI are usually performed through direct manipulation of the graphical elements. In addition to computers, GUIs can be found in hand-held devices such as MP3 players, portable media players, gaming devices and smaller household, office and industry equipment. The GUIs may be provided in a software, a software application, a web browser, etc. The GUIs may be displayed on a user device (e.g., mobile device, wearable device). The GUIs may be provided through a mobile application.

In some cases, notification or alert may be generated upon detection of an event (e.g., presence of foaming, prediction of foaming). A notification about the detection result may be generated and provided to a user. Such output result may be delivered to the user via any suitable approach or be in any suitable form, such as audio, visual, or tactile feedback. The notification or output result may be delivered through a user device. The notification may comprise an alert that can be delivered in any suitable forms (e.g., audio, visual alert in a GUI, webhooks that can be integrated into other applications, etc) or via any suitable communication channels (e.g., email, Slack, SMS). In some cases, the output result may also be delivered to any entities that are monitoring the experiment run or fermentation process. Alternatively or in addition to, the output result or notification may be delivered periodically like a report.

The monitoring system 800 may include or be in communication with an electronic display 835 that comprises a user interface (UI) 840 for providing, for example, displaying GUIs provided by the user interface (UI) module 808. Examples of UI's include, without limitation, a graphical user interface (GUI) and web-based user interface.

The monitoring system 800 may be in communication with a user device that comprises the electronic display 835 rendering the UI 840. Examples of user devices may include, but are not limited to, mobile devices, smartphones/cellphones, tablets, personal digital assistants (PDAs), laptop or notebook computers, desktop computers, media content players, television sets, video gaming station/system, virtual reality systems, augmented reality systems, microphones, or any electronic device capable of analyzing, receiving, providing or displaying certain types of feedback data (e.g., receiving user input, delivering alert, etc) to a user.

The electronic display 835 may be a screen. The display may or may not be a touchscreen. The display may be a light-emitting diode (LED) screen, OLED screen, liquid crystal display (LCD) screen, plasma screen, or any other type of screen. The display may be configured to show a user interface (UI) or a graphical user interface (GUI) rendered through an application (e.g., via an application programming interface (API) executed on the user device).

The monitoring system 800, fermentation system 850, and/or user device may be connected or interconnected to one or more databases 820. The databases may be one or more memory devices configured to store data. Additionally, the databases may also, in some embodiments, be implemented as a computer system with a storage device. In one aspect, the databases may be used by components of the network layout to perform one or more operations consistent with the disclosed embodiments. One or more local databases, and cloud databases of the platform may utilize any suitable database techniques. For instance, structured query language (SQL) or “NoSQL” database may be utilized for storing the image data, sensor data, historical data (e.g., experiment data), training datasets, predictive model or algorithms. Some of the databases may be implemented using various standard data-structures, such as an array, hash, (linked) list, struct, structured text file (e.g., XML), table, JavaScript Object Notation (JSON), NOSQL and/or the like. Such data-structures may be stored in memory and/or in (structured) files. In another alternative, an object-oriented database may be used. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. In some embodiments, the database may include a graph database that uses graph structures for semantic queries with nodes, edges and properties to represent and store data. If the database of the present invention is implemented as a data-structure, the use of the database of the present invention may be integrated into another component such as the component of the present invention. Also, the database may be implemented as a mix of data structures, objects, and relational structures. Databases may be consolidated and/or distributed in variations through standard data processing techniques. Portions of databases, e.g., tables, may be exported and/or imported and thus decentralized and/or integrated.

In some embodiments, the monitoring system 800 may construct the database for fast and efficient data retrieval, query and delivery. For example, the monitoring system 800 may provide customized algorithms to extract, transform, and load (ETL) the data. In some embodiments, the monitoring system 800 may construct the databases using proprietary database architecture or data structures to provide an efficient database model that is adapted to large scale databases, is easily scalable, is efficient in query and data retrieval, or has reduced memory requirements in comparison to using other data structures.

In one embodiment, the databases may comprise storage containing a variety of data consistent with disclosed embodiments. For example, the databases may store, for example, raw data collected from the fermentation system, sensors, training datasets, data about a trained predictive model (e.g., parameters, hyper-parameters, model architecture, training dataset, performance metrics, threshold, rules, etc), data generated by a trained model (e.g., output of a model, latent features, input and output of a component of the model system, etc), predictive models, algorithms, and the like. In certain embodiments, one or more of the databases may be co-located with the monitoring system (e.g., server), may be co-located with one another on the network, or may be located separately from other devices. One of ordinary skill will recognize that the disclosed embodiments are not limited to the configuration and/or arrangement of the database(s).

In some embodiments, the monitoring system 800 may be hosted on a server 810. In some embodiments, the monitoring system may be implemented as a hardware accelerator, software executable by a processor and various others. In some embodiments, the monitoring system may employ an edge intelligence paradigm that data processing and prediction is performed at the edge or edge gateway. In some cases, one or more of the trained model as disclosed herein may be built, developed and trained on the cloud/data center and run on the fermentation system (e.g., hardware accelerator) for inference. For example, the autoencoder and the classifier system may be pre-trained on the cloud and transmitted to the fermentation system 850 for implementation, then the continual training of the autoencoder and/or the classifier system may be performed on the cloud as new sensor data are collected. In such cases, a fixed model may be implemented in the fermentation system with the training and further tuning of the model performed on the cloud. Sensor data may be transmitted to the remote server 810 which are used to update the model and the updated model (e.g., parameters of the model that are updated) may be downloaded to the fermentation system (e.g., control module of the fermentation system) for implementation. Sensor data for the continual training may be transmitted to the remote server periodically or according to a pre-determined transmission rule (e.g., frequency for data transmission, event that triggers a data transmission (e.g., user command requesting an update, detection of data drift that triggers an update). In alternative cases, a machine learning model or one or more components of the model may be pre-trained on the server 820 and the continual training may be performed on the edge device (e.g., fermentation system). For example, the autoencoder and the classifier may be pre-trained on the cloud and transmitted to the fermentation system 850 for implementation. A continual training of the autoencoder and/or classifier may be performed on the fermentation system 850 with the newly collected sensor data. In such cases, a pre-trained model may be implemented in the fermentation system 850 with further tuning of the model performed on the local device. Maintaining close proximity to the edge devices (e.g., sensor, fermentation system, bioreactor bay) rather than sending all data to a distant centralized cloud, helps to minimize latency allowing for maximum performance, faster response times, and more effective maintenance and operational strategies. It may also significantly reduce overall bandwidth requirements and the cost of managing widely distributed networks.

In some cases, at least a portion of data processing may be performed at the edge (i.e., local fermentation system). Raw sensor data collected at the edge device or fermentation system 850 may be pre-processed locally before sending to the cloud. For example, the input data pre-processing module to provide functions such as ingesting of sensor data into a local storage repository (e.g., local time-series database), data cleansing, data enrichment (e.g., decorating data with metadata), data alignment, data annotation, data tagging, or data aggregation may be performed at the fermentation system. The pre-processed sensor data may then be transmitted to the server 810 for training or updating a model. In some cases, the software running on the fermentation computer system may be configured to aggregate the raw data across a time duration (e.g., about 1, 2, 3, 4, 5, 6, 7, 8, 9, 10 seconds, about 1, 2, 3, 4, 5, 6, 7, 8, 9, 10 minutes, about 1, 2, 3, 4, 5, 6, 7, 8, 9, 10 hours, etc), across data types (e.g., video data, temperature data, pH, OD, etc) or sources and sent to a remote entity (e.g., third-party application server, remote server, etc) as a package.

Network 830 may be a network that is configured to provide communication between the various components illustrated in FIG. 8. The network may be implemented, in some embodiments, as one or more networks that connect devices and/or components in the network layout for allowing communication between them. For example, fermentation system, sensors, monitoring system, user device and database may be in operable communication with one another over network 830. Direct communications may be provided between two or more of the above components. The direct communications may occur without requiring any intermediary device or network. Indirect communications may be provided between two or more of the above components. The indirect communications may occur with aid of one or more intermediary device or network. For instance, indirect communications may utilize a telecommunications network. Indirect communications may be performed with aid of one or more router, communication tower, satellite, or any other intermediary device or network. Examples of types of communications may include, but are not limited to: communications via the Internet, Local Area Networks (LANs), Wide Area Networks (WANs), Bluetooth, Near Field Communication (NFC) technologies, networks based on mobile data protocols such as General Packet Radio Services (GPRS), GSM, Enhanced Data GSM Environment (EDGE), 3G, 4G, 5G or Long Term Evolution (LTE) protocols, Infra-Red (IR) communication technologies, and/or Wi-Fi, and may be wireless, wired, or a combination thereof. In some embodiments, the network may be implemented using cell and/or pager networks, satellite, licensed radio, or a combination of licensed and unlicensed radio. The network may be wireless, wired, or a combination thereof.

The monitoring system or one or more components of the monitoring system may be implemented by a computer system that may comprise a laptop computer, a desktop computer, a central server, distributed computing system, etc. The processor may be a hardware processor such as a central processing unit (CPU), a graphic processing unit (GPU), a general-purpose processing unit, which can be a single core or multi core processor, or a plurality of processors for parallel processing. The processor can be any suitable integrated circuits, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512 bit, 256 bit, 128 bit, 64 bit, 32 bit, or 16 bit data operations.

The various functions, algorithms, methods performed or supported by the monitoring system such as parameter estimation, continual training, data processing, executing a trained model and the like may be implemented in software, hardware, firmware, embedded hardware, standalone hardware, application specific-hardware, or any combination of these. The state estimation module, foam detection and predicting module, input data pre-processing module and techniques described herein may be realized in digital electronic circuitry, integrated circuitry, specially designed ASICs (application specific integrated circuits), computer hardware, firmware, software, and/or combinations thereof. These systems, devices, and techniques may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device. These computer programs (also known as programs, software, software applications, or code) may include machine instructions for a programmable processor, and may be implemented in a high-level procedural and/or object-oriented programming language, and/or in assembly/machine language. As used herein, the terms “machine-readable medium” and “computer-readable medium” refer to any computer program product, apparatus, and/or device (such as magnetic discs, optical disks, memory, or Programmable Logic Devices (PLDs)) used to provide machine instructions and/or data to a programmable processor.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method for foam control for a fermentation system comprising:
obtaining image data from an imaging device located at the fermentation system; and
processing the image data using a first machine learning algorithm trained model to generate an output indicating a presence of foam or a level of foaming within a bioreactor of the fermentation system.

2. The method of claim 1, wherein the fermentation system comprises a plurality of the bioreactors configured to receive a fermentation agent and a controller for controlling one or more components of each of the plurality of the bioreactors.

3. The method of claim 2, wherein the one or more components are controlled based on a control instruction generated based at least in part on the presence of foam or the level of foaming.

4. The method of claim 1, wherein the image data contains at least a portion of a content in the bioreactor.

5. The method of claim 1, further comprising generating a control signal to adjust an operational status of the bioreactor based on the presence of foam or the level of foaming.

6. The method of claim 5, wherein the operational status is selected from airflow, pressure, temperature, or an agitation speed within the bioreactor.

7. The method of claim 1, further comprising predicting, using a second machine learning algorithm trained model, a state of a fermentation process within the bioreactor based at least in part on the image data and one or more real-time parameters.

8. The method of claim 7, wherein the one or more real-time parameters are estimated using sensor data.

9. The method of claim 8, wherein at least a portion of the sensor data is not a direct measurement of the one or more real-time parameters.

10. The method of claim 7, wherein the one or more real-time parameters are pH, dissolved oxygen tension, optical density, or temperature.

11. The method of claim 1, wherein the fermentation system comprises a robotic component configured to provide a fermentation agent to the bioreactor.

12. A system for foam control in a fermentation system, the system comprising:
an imaging device located at the fermentation system, wherein the imaging device is configured to capture image data containing at least a portion of a bioreactor of the fermentation system; and
one or more processors configured to:
receive the image data and process the image data using a first machine learning algorithm trained model to generate an output indicating a presence of foam or a level of foaming within the bioreactor.

13. The system of claim 12, wherein the fermentation system comprises a plurality of the bioreactors configured to receive a fermentation agent and a controller for controlling one or more components of each of the plurality of the bioreactors, wherein the one or more components are controlled based on a control instruction generated based at least in part on the presence of foam or the level of foaming.

14. (canceled)

15. The system of claim 12, wherein the image data contains at least a portion of a content in the bioreactor.

16. The system of claim 12, wherein the one or more processors are configured to further generate a control command to adjust an operational status of the bioreactor based on the presence of foam or the level of foaming.

17. The system of claim 16, wherein the operational status is selected from the group consisting of airflow, pressure, temperature and agitation speed within the bioreactor.

18. The system of claim 12, wherein the one or more processors are configured to further predict, using a second machine learning algorithm trained model, a state of a fermentation process within the bioreactor based at least in part on the image data and one or more real-time parameters, wherein the one or more real-time parameters are estimated using sensor data.

19. (canceled)

20. The system of claim 18, wherein at least a portion of the sensor data is not a direct measurement of the one or more real-time parameters.

21. The system of claim 18, wherein the one or more real-time parameters are selected from the group consisting of pH, dissolved oxygen tension, optical density and temperature.

22. The system of claim 12, wherein the fermentation system comprises a robotic component configured to provide a fermentation agent to the bioreactor.
